(19) United States
(12) Patent Application Publication
(10) Pub. No.: US 2011/0230877 A1
(43) Pub. Date: Sep. 22, 2011

(54) Pulsed Electric Field Probe for Glaucoma Surgery

(75) Inventors: John Huculak, Mission Viejo, CA (US); Steven Kovalcheck, Aliso Viejo, CA (US); Casey Lind, Irvine, CA (US)

(73) Assignee: Alcon Research, LTD., Fort Worth, TX (US)

(21) Appl. No.: 12/725,020

(22) Filed: Mar. 16, 2010

(51) Int. Cl. A61B 18/14

(52) U.S. Cl. 606/41

(57) ABSTRACT

A small gauge pulsed electric field/aspirator probe. The probe has a generally cylindrical cannula with a generally smooth distal end. A port is located near a distal end of the cannula on a side of the cannula. A pair of electrodes is located at the port. An electric pulse generator is coupled to the pair of electrodes. A distance between the distal end of the cannula and the port is approximately equal to the distance between a back wall of Schlemm's canal and a trabecular meshwork in a human eye. The electric pulse generator applies a pulsed electric field to the pair of electrodes sufficient to dissociate protein bonds that hold the trabecular meshwork together.

### BACKGROUND OF THE INVENTION

The present invention relates to glaucoma surgery and more particularly to a method and device for performing glaucoma surgery using a small gauge pulsed electric field/aspirator probe with a retractable pick.

Glaucoma, a group of eye diseases affecting the retina and optic nerve, is one of the leading causes of blindness worldwide. Glaucoma results when the intraocular pressure (IOP) increases to pressures above normal for prolonged periods of time. IOP can increase due to an imbalance of the production of aqueous humor and the drainage of the aqueous humor. Left untreated, an elevated IOP causes irreversible damage the optic nerve and retinal fibers resulting in a progressive, permanent loss of vision.

The eye's ciliary body epithelium constantly produces aqueous humor, the clear fluid that fills the anterior chamber of the eye (the space between the cornea and iris). The aqueous humor flows out of the anterior chamber through the uveoscleral pathways, a complex drainage system. The delicate balance between the production and drainage of aqueous humor determines the eye's IOP.

Open angle (also called chronic open angle or primary open angle) is the most common type of glaucoma. With this type, even though the anterior structures of the eye appear normal, aqueous fluid builds within the anterior chamber, causing the TOP to become elevated. Left untreated, this may result in permanent damage of the optic nerve and retina. Eye drops are generally prescribed to lower the eye pressure. In some cases, surgery is performed if the IOP cannot be adequately controlled with medical therapy.

Only about 10% of the population suffers from acute angle closure glaucoma. Acute angle closure occurs because of an abnormality of the structures in the front of the eye. In most of these cases, the space between the iris and cornea is more narrow than normal, leaving a smaller channel for the aqueous to pass through. If the flow of aqueous becomes completely blocked, the IOP rises sharply, causing a sudden angle closure attack.

Secondary glaucoma occurs as a result of another disease or problem within the eye such as: inflammation, trauma, previous surgery, diabetes, tumor, and certain medications. For this type, both the glaucoma and the underlying problem must be treated.

FIG. 1 is a diagram of the front portion of an eye that helps to explain the processes of glaucoma. In FIG. 1, representations of the lens 110, cornea 120, iris 130, ciliary bodies 140, trabecular meshwork 150, and Schlemm's canal 160 are pictured. Anatomically, the anterior chamber of the eye includes the structures that cause glaucoma. Aqueous fluid is produced by the ciliary bodies 140 that lie beneath the iris 130 and adjacent to the lens 110 in the anterior chamber. This aqueous humor washes over the lens 110 and iris 130 and flows to the drainage system located in the angle of the anterior chamber. The angle of the anterior chamber, which extends circumferentially around the eye, contains structures that allow the aqueous humor to drain. The first structure, and the one most commonly implicated in glaucoma, is the trabecular meshwork 150. The trabecular meshwork 150 extends circumferentially around the anterior chamber in the angle. The trabecular meshwork 150 seems to act as a filter, limiting the outflow of aqueous humor and providing a back pressure producing the IOP. Schlemm's canal 160 is located beyond the trabecular meshwork 150. Schlemm's canal 160 has collector channels that allow aqueous humor to flow out of the anterior chamber. The two arrows in the anterior chamber of FIG. 1 show the flow of aqueous humor from the ciliary bodies 140, over the lens 110, over the iris 130, through the trabecular meshwork 150, and into Schlemm's canal 160 and its collector channels.

If the trabecular meshwork becomes malformed or malfunctions, the flow of aqueous humor out of the anterior chamber can be restricted resulting in an increased IOP. The trabecular meshwork may become clogged or inflamed resulting in a restriction on aqueous humor flow. The trabecular meshwork, thus, sometimes blocks the normal flow of aqueous humor into Schlemm's canal and its collector channels.

Surgical intervention is sometimes indicated for such a blockage. Numerous surgical procedures have been developed to either remove or bypass the trabecular meshwork. The trabecular meshwork can be surgically removed by cutting, ablation, or by means of a laser. Several stents or conduits are available that can be implanted through the trabecular meshwork in order to restore a pathway for aqueous humor flow. Each of these surgical procedures, however, has drawbacks.

One approach that does not have the drawbacks of existing procedures involves using a pulsed electric field probe to remove trabecular meshwork tissue. Pulsed electric fields can be used to temporarily dissociate the protein bonds between trabecular meshwork tissue. While dissociated, the tissue can be aspirated through a lumen. A small gauge device with electrodes can be guided into Schlemm's canal and moved in a forward motion following the curvature of the trabecular meshwork. The motion causes the trabecular meshwork to be fed into the electrode port of the device, dissociating and removing the trabecular meshwork blocking the outflow of the aqueous humor.

### SUMMARY OF THE INVENTION

In one embodiment consistent with the principles of the present invention, the present invention is a small gauge pulsed electric field/aspirator probe. The probe has a generally cylindrical cannula with a distal end that defines a generally planar surface. A port is located near a distal end of the cannula. A retractable pick is located on the distal end of the cannula. A pair of electrodes is located at the port. An electric pulse generator is coupled to the pair of electrodes. A distance between the generally planar surface of the distal end of the cannula and the port is approximately equal to the distance between a back wall of Schlemm's canal and a trabecular meshwork in a human eye. The electric pulse generator applies a pulsed electric field to the pair of electrodes sufficient to dissociate protein bonds that hold the trabecular meshwork together

In another embodiment consistent with the principles of the present invention, the present invention is a small gauge pulsed electric field/aspirator probe. The probe has a generally cylindrical cannula with a generally smooth distal end. A port is located near a distal end of the cannula on a side of the cannula. A pair of electrodes is located at the port. An electric pulse generator is coupled to the pair of electrodes. A distance between the distal end of the cannula and the port is approximately equal to the distance between a back wall of Schlemm's canal and a trabecular meshwork in a human eye. The electric pulse generator applies a pulsed electric field to the pair of electrodes sufficient to dissociate protein bonds that hold the trabecular meshwork together.

In another embodiment consistent with the principles of the present invention, the present invention is a method of dissociating and removing trabecular meshwork from a human eye. The method comprises providing a pulsed electric field/aspirator probe with a generally cylindrical cannula, a port located near a distal end of the cannula on a side of the cannula, and a pair of electrodes located at the port, such that the location of the port on the cannula facilitates the placement of the port at the trabecular meshwork of a human eye; applying a pulsed electric field to the pair of electrodes so that the trabecular meshwork is dissociated without damaging the outer wall of Schlemm's canal; and aspirating the dissociated trabecular meshwork from the eye.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the front portion of an eye.

FIG. 2 is a perspective view of a small gauge pulsed electric field/aspirator probe according to the principles of the present invention.

FIG. 3 is a diagram of a small gauge pulsed electric field/aspirator probe system according to the principles of the present invention.

FIG. 4 is a block diagram of the functional elements of an exemplary pulse generator according to the principles of the present invention.

FIGS. 5A and 5B are perspective views of the distal end of a pulsed electric field/aspirator probe according to the principles of the present invention.

FIG. 6 is a diagram of one section of the port at the distal end of a pulsed electric field/aspirator probe according to the principles of the present invention.

FIG. 7 is a front view of the port at the distal end of a pulsed electric field/aspirator probe according to the principles of the present invention.

FIGS. 8 and 9 are views of a small gauge pulsed electric field/aspirator probe as used in glaucoma surgery.

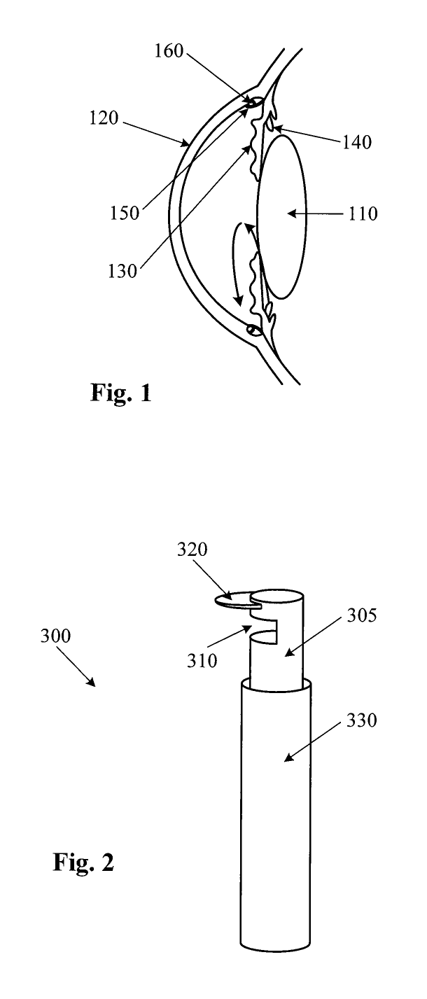

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

FIG. 2 is a perspective view of a small gauge pulsed electric field/aspirator probe according to the principles of the present invention. In the embodiment of FIG. 2, a cannula 305 includes port 310. A pick 320 is located on a distal end of cannula 305. Pick 320 may be fixed or retractable. If retractable, pick 320 retracts inside cannula 305. Port 310 includes one or more pairs of electrodes as more fully described herein. An irrigation sleeve 330 surrounds cannula 305. Irrigation fluid flows in the space between the outside of cannula 305 and the inside of irrigation sleeve 330.

Pick 320 is adapted to fit into Schlemm's canal so that the pulsed electric field probe can be used to dissociate and remove the trabecular meshwork (through aspiration provided through port 310). Pick 320 is a short protrusion that extends outward from the distal tip of cannula 305 in the direction of port 310. In one embodiment of the present invention, pick 320 has a sharp end that can be used to pierce the trabecular meshwork so that pick 320 can be placed in Schlemm's canal. In another embodiment of the present invention, pick 320 is optional. While pick 320 facilitates entry into Schlemm's canal, once port 310 is located on the trabecular meshwork, pick 320 is largely unnecessary. As such, pick 320 may be retracted into cannula 305. In other embodiments of the present invention, pick 320 is not present.

A high intensity pulsed electric field is provided at port 310 which is located along the trabecular meshwork (as best seen in FIG. 9). The distance between port 310 and the distal end of cannula 520 determines the location of port 310 in relation to the back wall of Schlemm's canal. This distance is such that port 310 is located at the trabecular meshwork (preferably the distance from the distal end of cannula 305 to the center of port 310 is equal to the distance between the trabecular meshwork and the back wall of Schlemm's canal). Locating port 310 at the trabecular meshwork ensures effective removal of it.

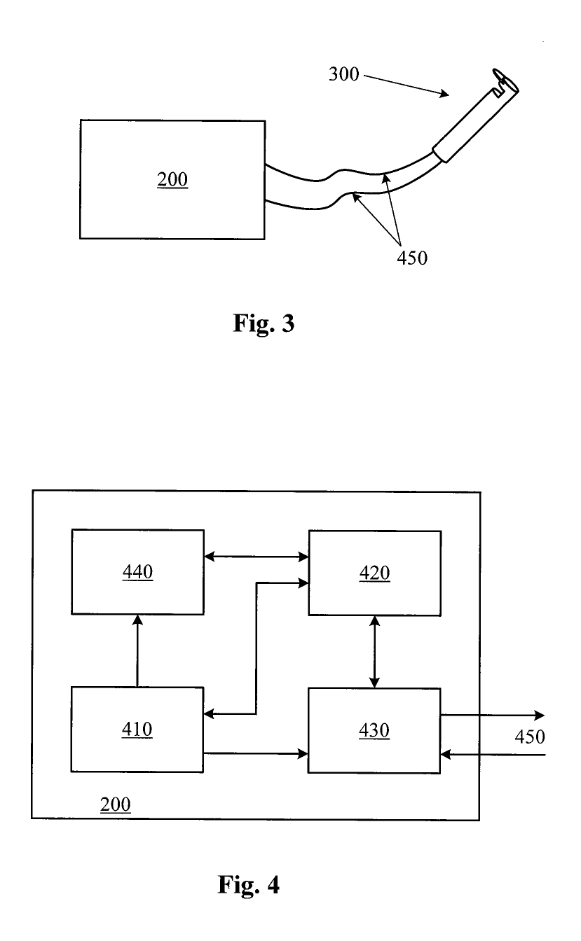

FIG. 3 illustrates the components of an exemplary small gauge pulsed electric field/aspirator probe according to some embodiments of the invention. The system includes a pulsed electric field aspirator probe 300 and pulse generator 200. Pulse generator 200 produces high intensity pulses for application to the eye through electrode lead wires 450.

FIG. 4 illustrates functional elements of a pulse generator 200 according to some embodiments of the present invention. Pulse generator 200 includes a main power supply 410, which may be operated from an external alternating current source (e.g., 120 volts at 60 Hz) or direct current source. HIPEF (high intensity pulsed electric field) pulse generator 430 generates the high intensity pulses, from the main power supply 410, under the control of control circuit 420. The high intensity pulses are supplied to electrodes in port 310 of pulsed electric field/aspirator probe 300 through lead wires 450. User interface 440 provides the operator with appropriate mechanisms for operating the pulse generator 200 (e.g., switches, touch-screen inputs, or the like), as well as appropriate feedback (e.g., device status, etc.). Further details of a high intensity electric pulse generator apparatus that can readily be adapted for the present application are provided in U.S. Patent Application Publication 2007/0156129 A1, published 5 Jul. 2007, the entire contents of which are incorporated herein by reference.

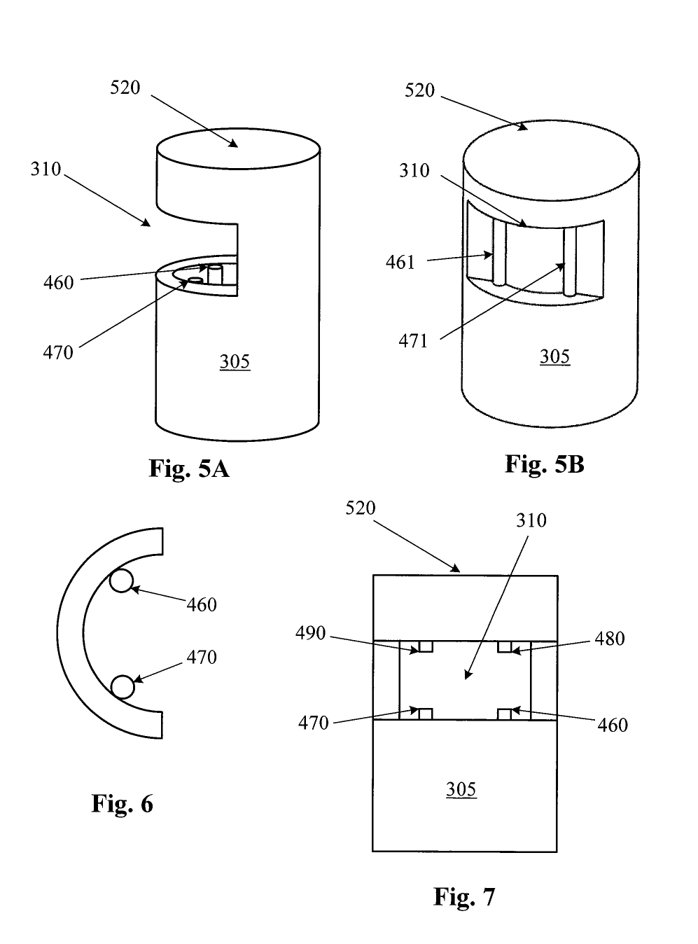

FIGS. 5A and 5B are perspective views of the distal end of a pulsed electric field/aspirator probe according to the principles of the present invention. FIG. 6 is a diagram of one section of the port at the distal end of a pulsed electric field/aspirator probe according to the principles of the present invention. FIG. 7 is a view of the port at the distal end of a pulsed electric field/aspirator probe according to the principles of the present invention. FIGS. 5A, 5B, 6 and 7 show possible locations of electrodes in port 310. In these FIGS. 5A, 6 and 7, two pairs of electrodes are depicted. While in FIG. 5B, a single parallel pair of electrodes is depicted.

As shown in FIG. 7, a first pair of electrodes includes electrodes 460 and 480. A second pair of electrodes includes electrodes 470 and 490. The two pairs of electrodes (460 and 480, 470 and 490) are located in port 310. In one example (as seen in FIGS. 5 and 6), electrodes 460 and 470 are located inside cannula 305 at the bottom lip of port 310. Electrodes 460 and 470 are spaced apart from each other so as to provide an electric field in the vicinity of port 310. In this example, electrodes 480 and 490 are located on the upper lip of port 310 generally opposite electrodes 460 and 470, respectively. While electrode pairs are shown opposite each other in this example, in other examples, electrode pairs need not be located opposite each other. For example, any combination of electrodes can form an electrode pair (460 and 490, 470 and 480, 460 and 470, 480 and 490). Electrodes may also be located outside cannula 305 (and inside or outside the irrigation sleeve, if present). Alternatively, the electrodes may be embedded in cannula 305 or the irrigation sleeve (if present). While four electrodes are shown, any number of electrodes may be used. Alternatively the electrodes 461 and 471 may be located in parallel on either side of port 310 as illustrated in FIG. 5B.

In the example of FIGS. 5-7, the electrodes (460, 470, 461, 471, 480, and 490) are located such that the electric field emitted from them is most intense at the port 310. In this manner, the electric field at port 310 is such that trabecular meshwork tissue is dissociated when it enters port 310. It can then be aspirated through the lumen of cannula 305. Since an the strength of an electric field decreases in proportion to the square of the distance from an emitting electrode, the field at port 310 can be controlled such that the electric field acts on the tissue that is located in port 310 and not on tissues located near cannula 305.

In operation, pulse generator 200 produces high intensity pulses for application to electrode pairs 460 and 480, 461 and 471 (and 470 and 490). The high frequency pulses produce an electric field that originates between the selected electrode. By selecting different electrodes, pulsed electric fields can be applied to tissue from any of the electrodes. These pulsed electric fields are such that the affected tissue is dissociated. Once dissociated, the tissue can be aspirated through the interior of cannula 305. As more fully described in U.S. Patent Application Publication 2007/0156129 A1, the pulsed electric fields are of a strength and duration to dissociate the proteinaceous bonds that hold tissue together. As opposed to ablation or other techniques that involve burning tissue, the application of pulsed electric fields in the manner consistent with the present invention involves the dissociation of the bonds that hold the tissue together.

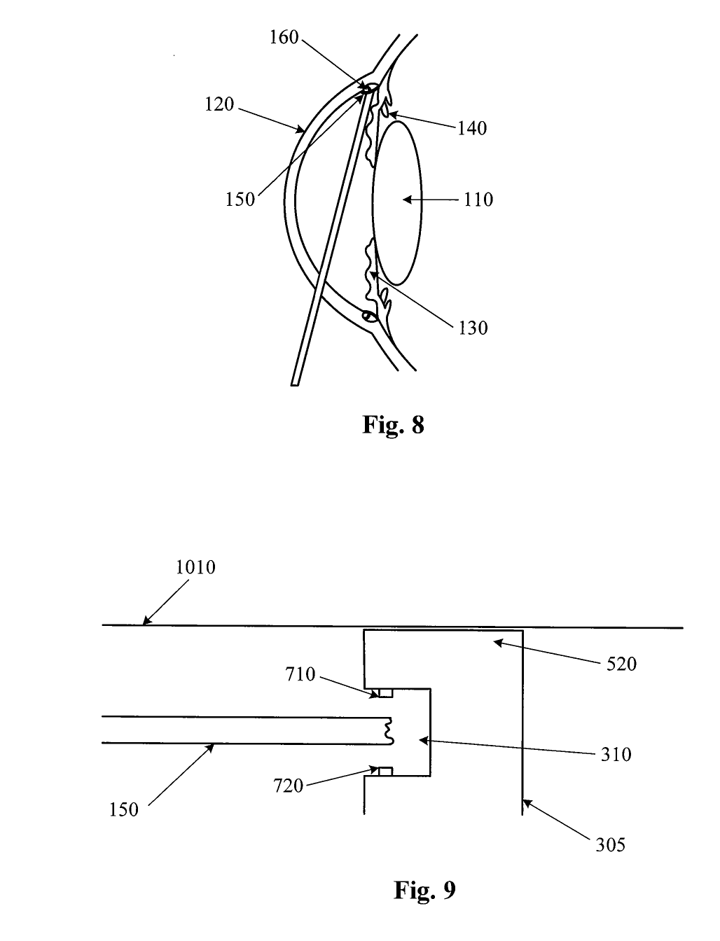

FIGS. 8 and 9 are views of a small gauge pulsed electric field/aspirator probe as used in glaucoma surgery. In FIG. 8, cannula 305 is inserted through a small incision in the cornea 120. The distal end of cannula 305 (the end that has port 310) is advanced through the angle to the trabecular meshwork 150. The retractable pick is extended so that an opening can be made in the trabecular meshwork. The retractable pick is then retracted so as to avoid damaging a wall of Schlemm's canal 160. The distal end of cannula 305 is then advanced through the opening in the trabecular meshwork 150 and into Schlemm's canal 160. In this position, port 310 is located at the trabecular meshwork 150. High intensity pulsed electric fields can then be applied to the electrodes to remove the trabecular meshwork 150 from the eye.

FIG. 9 is an exploded view of the location of the distal end of cannula 305 during the removal of the trabecular meshwork 150 (note that in this position, the retractable pick is in a retracted position). In this position, port 310 is located at the trabecular meshwork 150. Cannula 305 is then advanced in the direction of port 310 to dissociate and remove the trabecular meshwork 150. Cannula 305 is advanced through an arc in one direction, port 310 is then rotated 180 degrees, and cannula 305 is then advanced in an arc in the other direction. In this manner, the distal end of cannula 305 (and port 310) is moved in an arc around the circumference of the angle to remove a substantial portion of the trabecular meshwork through a single corneal incision. If desired, a second corneal incision opposite the first corneal incision can be made so that the cannula 305 can be swept through a second arc of the angle. In this manner, either through one or two corneal incisions, a significant portion of the trabecular meshwork can be removed by the pulsed electric field/aspirator probe.

A shown in FIG. 9, the distal end 520 of cannula 305 is located adjacent the back wall of Schlemm's canal 1010. In this manner, the distance between the distal end 520 of cannula 305 and the port 310 is approximately equal to the distance between the trabecular meshwork 150 and the back wall of Schlemm's canal 1010 (approximately 0.3 millimeters). Electrodes 710 and 720 are located on opposite sides of the trabecular meshwork 150 so that a field generated between electrodes 710 and 720 dissociate the protein bonds that hold the trabecular meshwork 150 together. When dissociated, the trabecular meshwork can then be aspirated through port 310. Moreover, the location of electrodes 710 and 720 are such that the electric field acts on the tissue in the port 310 (i.e. dissociates the trabecular meshwork 150) without damaging the back wall of Schlemm's canal 1010.

From the above, it may be appreciated that the present invention provides a system and methods for performing glaucoma surgery with a small gauge pulsed electric field/aspirator probe. The present invention provides a small gauge pulsed electric field/aspirator probe with an optional pick that can be advanced into Schlemm's canal to dissociate and aspirate the trabecular meshwork. Methods of using the probe are also disclosed. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pulsed electric field/aspirator probe comprising:
a generally cylindrical cannula, the cannula having a distal end that defines a generally planar surface;
a port located near a distal end of the cannula;
a retractable pick located on the distal end of the cannula;
a pair of electrodes located at the port;
an electric pulse generator coupled to the pair of electrodes;
wherein a distance between the generally planar surface of the distal end of the cannula and the port is approximately equal to the distance between a back wall of Schlemm's canal and a trabecular meshwork in a human eye;
and further wherein the electric pulse generator applies a pulsed electric field to the pair of electrodes sufficient to dissociate protein bonds that hold the trabecular meshwork together.

2. The probe of claim 1 wherein the retractable pick further comprises a sharp edge for piercing the trabecular meshwork.

3. The probe of claim 1 further comprising:
a second set of electrodes.

4. The probe of claim 1 wherein the electrodes are on an interior of the cannula.

5. The probe of claim 1 further comprising:
an irrigation sleeve surrounding the cannula.

6. The probe of claim 1 wherein the cannula has a diameter between about 0.25 and 0.36 millimeters.

7. The probe of claim 1 wherein the distance between the generally planar surface of the distal end of the cannula and the port is approximately 0.3 millimeters.

8. The probe of claim 1 wherein tissue is aspirated through the port.

9. The probe of claim 1 wherein the retractable pick is made of nitinol.

10. A pulsed electric field/aspirator probe comprising:
a generally cylindrical cannula with a generally smooth distal end;
a port located near a distal end of the cannula on a side of the cannula;
a pair of electrodes located at the port;
an electric pulse generator coupled to the pair of electrodes
wherein a distance between the distal end of the cannula and the port is approximately equal to the distance between a back wall of Schlemm's canal and a trabecular meshwork in a human eye;
and further wherein the electric pulse generator applies a pulsed electric field to the pair of electrodes sufficient to dissociate protein bonds that hold the trabecular meshwork together.

11. The probe of claim 10 wherein the distal end of the cannula is configured to rest against the outer wall of Schlemm's canal.

12. The probe of claim 10 further comprising:
a second set of electrodes.

13. The probe of claim 10 wherein the distal end of the cannula has a diameter between about 0.25 and 0.36 millimeters.

14. The probe of claim 10 wherein the distance between the distal end of the cannula and the port is approximately 0.3 millimeters.

15. The probe of claim 10 wherein tissue is aspirated through the port.

16. The probe of claim 10 further comprising:
an irrigation sleeve surrounding the cannula.

17. A method of dissociating and removing trabecular meshwork from a human eye, the method comprising:
providing a pulsed electric field/aspirator probe with a generally cylindrical cannula, a port located near a distal end of the cannula on a side of the cannula, and a pair of electrodes located at the port, such that the location of the port on the cannula facilitates the placement of the port at the trabecular meshwork of a human eye;
applying a pulsed electric field to the pair of electrodes so that the trabecular meshwork is dissociated without damaging the outer wall of Schlemm's canal; and
aspirating the dissociated trabecular meshwork from the eye.

18. The method of claim 17 wherein aspirating the dissociated trabecular meshwork from the eye further comprises aspirating the dissociated trabecular meshwork through the port and through the cannula.

19. The method of claim 17 wherein the pulsed electric field/aspirator probe is provided with a retractable pick located on the distal end of the cannula.

20. The method of claim 19 further comprising:
extending the retractable pick so that an opening can be formed in the trabecular meshwork;
retracting the retractable pick; and
inserting the distal end of the cannula in Schlemm's canal.
